(19) United States
(12) Patent Application Publication
(10) Pub. No.: US 2025/0018207 A1
(43) Pub. Date: Jan. 16, 2025

(54) SENSATION MANAGEMENT

(71) Applicant: Medtronic, Inc., Minneapolis, MN (US)

(72) Inventors: Amy E. Thompson-Nauman, Ham Lake, MN (US); Matthew J. Hoffman, St. Paul, MN (US); Vladimir P. Nikolski, San Diego, MN (US); Mary M. Morris, Shoreview, MN (US)

(21) Appl. No.: 18/765,497

(22) Filed: Jul. 8, 2024

Related U.S. Application Data
(60) Provisional application No. 63/513,038, filed on Jul. 11, 2023.

(51) Int. Cl. A61N 1/39; A61N 1/05; A61N 1/32; A61N 1/36; A61N 1/365

(52) U.S. Cl. CPC A61N 1/39624; A61N 1/0551; A61N 1/327; A61N 1/36071; A61N 1/365; A61N 1/39622

(57) ABSTRACT

In some examples, a medical device includes an elongated structure configured to extend from an access point of a patient through an intercostal space within the patient. The medical device may further include a plurality of electrodes comprising a first subset of electrodes and a second subset of electrodes, where at least the first subset of electrodes is carried on a distal portion of the elongated structure. The first subset of electrodes of the plurality of electrodes may be configured to deliver electrical therapy to a heart of the patient or sense cardiac activity. The second subset of electrodes of the plurality of electrodes may be configured to deliver energy to tissue proximate a ribcage of the patient to reduce a sensation in one or more intercostal nerves.

### RELATED APPLICATIONS

This application claims the benefit of U.S. Provisional Application Ser. No. 63/513,038, filed Jul. 11, 2023, the entire contents of each of which are incorporated herein by reference.

### TECHNICAL FIELD

This disclosure relates to cardiac sensing and therapy and, more particularly, implanted systems for defibrillation, sensing, and/or pacing in extracardiac applications and for managing sensations associated with such systems.

### BACKGROUND

Implantable systems, such as pacemakers with or without cardioversion or defibrillation capabilities, may treat cardiac dysfunction, such as bradycardia, tachyarrhythmia, and heart failure. Such implantable systems may include electrical devices configured to deliver therapy via electrodes, often carried by one or more implantable medical leads.

Owing to the inherent surgical risks in attaching and replacing implantable medical leads directly within or on the heart, subcutaneous implantable systems have been devised, in which the implantable system and leads are located subcutaneously outside of the thorax. It has also been proposed that the distal portion of a lead of an implantable system may be implanted within the thorax, e.g., substernally or intercostally.

Implantable medical leads are also used to monitor and/or deliver therapies to tissues other than the heart. Implantable medical leads may be used to position one or more electrodes within or near target nerves, muscles, or organs to deliver electrical stimulation to such tissues. Implantable medical leads may be used to position one or more sensors within or near target tissue to monitor biological signals from such tissues.

### SUMMARY

Medical device systems, such as implantable medical device systems or partially implantable medical device systems, may result in the patient experiencing sensation (e.g., pain, paresthesia, etc.) due to components of the medical device systems being extended or navigated through tissue of the patient (e.g., tissue in the intercostal space between ribs). Post-thoracotomy pain syndrome (PTPS) has been observed in patients following thoracic surgery. Trauma from incision, retraction, instrument placement, or suturing may cause trauma to one or more intercostal nerves, which may contribute to PTPS. Pain associated with thoracic surgery may be acute and/or chronic. Additionally, medical device systems configured to provide electrical therapy using electrodes outside of the heart may also result in the patient experiencing sensation in tissue proximate the ribcage of the patient.

In accordance with the techniques of this disclosure, a medical device may be configured to deliver therapy (e.g., electrical energy or drug therapy) to tissue proximate the ribcage of the patient to reduce a sensation. In some examples, the medical device may be configured to deliver peripheral nerve stimulation, electroporation energy, or other therapy (including drug therapy) anterior to the heart to reduce or eliminate sensation in one or more intercostal nerves. The therapy delivered to the tissue and one or more intercostal nerves may reduce or eliminate the sensation associated with navigation of components of the medical device through the intercostal space or with electrical therapy delivered to the heart.

In some examples, a medical device includes an elongated structure configured to extend from an access point of a patient through an intercostal space within the patient. The medical device further includes a plurality of electrodes comprising a first subset of electrodes and a second subset of electrodes, where at least the first subset of electrodes is carried on a distal portion of the elongated structure. The first subset of electrodes of the plurality of electrodes is configured to deliver electrical therapy to a heart of the patient or sense cardiac activity. The second subset of electrodes of the plurality of electrodes is configured to deliver energy to tissue proximate a ribcage of the patient to reduce a sensation in one or more intercostal nerves.

In some examples, a method includes delivering, by a medical device and via a first subset of a plurality of electrodes, electrical therapy to a heart of a patient, wherein the first subset of electrodes of the plurality of electrodes is carried by a distal portion of an elongated structure extending from an access point of the patient through an intercostal space within the patient. The method further includes delivering, by the medical device and via a second subset of the plurality of electrodes of the medical device, energy to tissue proximate the ribcage of a patient to reduce a sensation in one or more intercostal nerves.

In some examples, a medical device includes an elongated structure configured to extend from an access point of a patient through an intercostal space within the patient. The medical device further includes a plurality of electrodes carried on a distal portion of the elongated structure, wherein at least a subset of electrodes of the plurality of electrodes is configured to at least deliver electrical therapy to a heart of the patient or sense cardiac activity. The distal portion of the elongated structure is configured to deliver a therapy agent to tissue proximate a ribcage of the patient, and wherein the therapy agent comprises at least one of an anti-inflammatory agent, an analgesic agent, a neurotoxin, or an antimicrobial agent.

This summary is intended to provide an overview of the subject matter described in this disclosure. It is not intended to provide an exclusive or exhaustive explanation of the systems, devices, and methods described in detail within the accompanying drawings and description below. Further details of one or more examples are set forth in the accompanying drawings and the description below. Other features, objects, and advantages will be apparent from the description and drawings, and from the statements provided below.

### BRIEF DESCRIPTION OF DRAWINGS

FIG. 1 is a conceptual drawing illustrating a front view of a patient implanted with an example implantable medical device system.

FIG. 2 is a conceptual drawing illustrating a perspective view of a patient implanted with an example implantable medical device system.

FIG. 3 is a conceptual drawing illustrating a side view of a patient implanted with an example implantable medical device system.

FIG. 4 is a conceptual drawing illustrating a side view of a patient implanted with an example implantable medical device system.

FIG. 5 is a conceptual drawing illustrating a side view of a patient implanted with an example implantable medical device system.

FIG. 6 is a conceptual drawing illustrating a side view of a patient implanted with an example implantable medical device system including a therapy agent.

FIG. 7A is a conceptual drawing illustrating a portion of an elongated structure with a therapy agent.

FIG. 7B is a conceptual drawing illustrating a portion of an elongated structure with a therapy agent.

FIG. 7C is a conceptual drawing illustrating a portion of an elongated structure with a therapy agent.

FIG. 7D is a conceptual drawing illustrating a portion of an elongated structure with a therapy agent.

FIG. 7C is a conceptual drawing illustrating a portion of an elongated structure with a therapy agent.

FIG. 7E is a conceptual drawing illustrating a portion of an elongated structure with a therapy agent.

FIG. 7F is a conceptual drawing illustrating a portion of an elongated structure with a therapy agent.

FIG. 8 is a conceptual drawing illustrating a perspective view of an example implantable medical device system with a delivery device.

FIG. 9 is a functional block diagram of an example configuration of electronic components of an example implantable medical device.

FIG. 10 conceptual diagram illustrating an example elongated structure carrying a shield.

FIG. 11 is a flow chart of an example technique for delivering therapy and energy to tissue via an example implantable medical device.

Like reference characters denote like elements throughout the description and figures.

### DETAILED DESCRIPTION

Implantable medical devices (IMDs), including and extravascular implantable cardioverter defibrillators (EV-ICDs) or other types of implantable cardioverter defibrillators (ICDs), may deliver cardiac pacing in addition to anti-tachyarrhythmia shocks. During implantation into a patient, one or more components of an IMD (e.g., elongated bodies, including medical leads) may be navigated through tissue of a patient. A patient may experience sensation (e.g., pain, paresthesia, etc.) in areas where nerves (e.g., intercostal nerves) are impinged by components of the IMD. Additionally, a patient may experience sensation during pacing delivered by an EV-ICD, such as via electrodes implanted substernally or intercostally. Sensation may be due to stimulation of skeletal muscles and intercostal nerves (and/or any other muscle tissue and nerve tissue) proximate the electrodes of the lead or on a medical device (e.g., a housing of an implantable medical device).

As described herein, medical devices including electrodes configured to deliver energy to tissue proximate the ribcage of the patient may reduce or eliminate the sensation in one or more intercostal nerves. Additionally, medical devices including therapy agents (e.g., an anti-inflammatory agent, an analgesic agent, a neurotoxin, or an antimicrobial agent, etc.) may reduce or eliminate the sensation in one or more intercostal nerves.

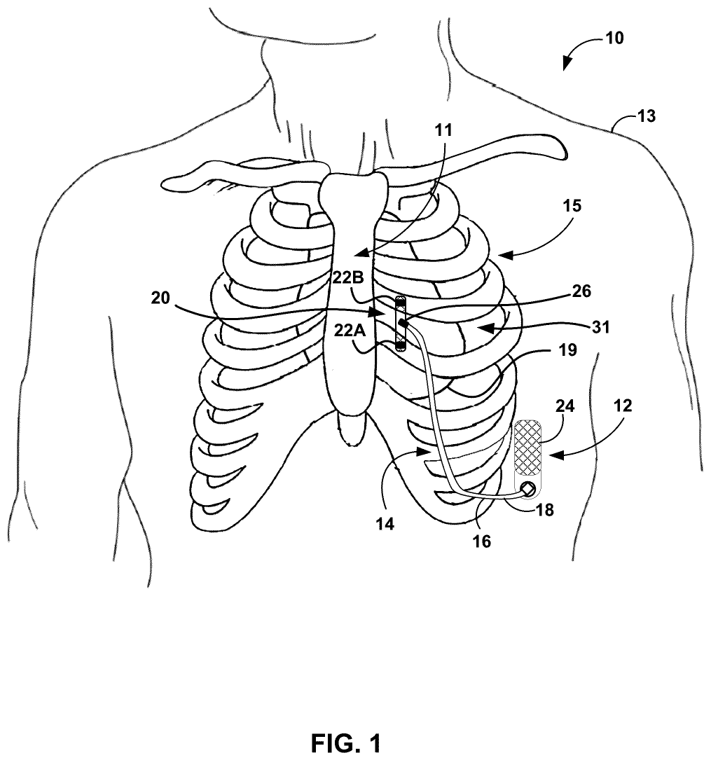

FIG. 1 illustrates an example medical system 10 (“system 10”) in accordance with techniques of this disclosure. System 10 is primarily described herein as an extravascular and/or extracardiac medical system, such as an EV-ICD system with a lead extending through an intercostal space 31 of a patient 13. However, it should be understood that the techniques of this disclosure may apply to other medical device systems. For example, the techniques of this disclosure may apply to a pacemaker configured to deliver pacing therapy but not defibrillation therapy. Additionally, it should be understood that the techniques of this disclosure may apply to non-cardiac devices (e.g., neurostimulators, pelvic and gastric devices, etc.). In general, the techniques of this disclosure may apply to any medical device or system that extends through tissue of a patient or delivers electrical therapy that may cause sensation. Additionally, although the example of FIG. 1 illustrates a lead extending through intercostal space 31, other lead locations and/or orientations are contemplated (e.g., a lead extending substernally).

System 10 may include a medical device, such as an IMD 12 (e.g., an ICD). IMD 12 may include a signal generator configured to provide cardiac pacing and/or defibrillation therapy. As shown in the example of FIG. 1, IMD 12 may be implanted subcutaneously on the left mid-axillary of a patient 13, superficially of the patient's ribcage 15 (also referred to herein as ribs 15 or one or more ribs 15). IMD 12 may be in wireless communication with an external device (not shown) (e.g., a computing device for use by a patient, a clinician, etc.) to transmit information to the external device. IMD 12 may be coupled to an elongated structure, such as an implantable medical lead 14 (“lead 14”). Although lead 14 is shown as a single lead in the example of FIG. 1, one or more leads or branching leads may be used in connection with IMD 12. Lead 14 may be configured to extend from an access point of patient 13 to an implantation site within patient 13. For example, as shown in the example of FIG. 1, lead 14 is configured to extend from an access point of patient 13 through intercostal space 31 between adjacent ribs 15 within patient 13. When extended through intercostal space 31 between adjacent ribs 15, a distal portion 20 of lead 14 may be positioned left of, right of, anterior of, and/or posterior of sternum 11. In other examples, lead 14 is configured to extend from an access point of patient 13 underneath sternum 11, for example, in a direction parallel to sternum 11.

Lead 14 may include a lead body 16 including a proximal portion 18 and distal portion 20, wherein lead 14 is configured to be implanted extra-thoracically (outside ribcage 15 and sternum 11, e.g., subcutaneously or submuscularly) or intra-thoracically (e.g., beneath ribs 15 and/or sternum 11) proximate a heart 19 of patient 13. In some examples, lead 14 extends subcutaneously to an intercostal space 31 (e.g., between adjacent ribs of ribs 15). In some examples, lead 14 extends subcutaneously toward the center of the torso of patient 13, for example, toward the xiphoid process of patient 13. While lead 14 is described primarily as an implantable medical lead, other bodies may be use in connection with the techniques of this disclosure, including but not limited to catheters, sheaths (e.g., introducer sheath), delivery devices, access tools, or other elongated tubular structures.

IMD 12 may include a housing 24 (e.g., a medical device housing) that forms a hermetic seal that protects components of IMD 12. Housing 24 of IMD 12 may be formed of a conductive material, such as titanium or titanium alloy, which may function as a housing electrode (sometimes referred to as a can electrode). In some embodiments, IMD 12 may be formed to include a plurality of electrodes on housing 24. IMD 12 also includes a connector assembly (also referred to as a connector block or header) that includes electrical feedthroughs through which electrical connections are made between conductors of lead 14 and electronic components included within housing 24 of IMD 12. IMD 12 may include multiple electrical feedthroughs (e.g., two or more) or connector assemblies in examples where multiple leads and/or multiple electrodes are separately connected to IMD 12. As will be described in further detail herein, housing 24 may house one or more processors, memories, transmitters, receivers, sensors, sensing circuitry, therapy circuitry, power sources and other appropriate components. Housing 24 may be configured to be implanted in a patient, such as patient 13. Alternatively, housing 24 may be external to patient 13 and connect to a proximal end of the lead that extends out of the body of the patient, e.g., through an incision.

In the example of FIG. 1, housing 24 of IMD 12 is implanted extrathoracically on the left side of the patient, e.g., under the skin and outside ribcage 15 (subcutaneously or submuscularly). Housing 24 of IMD 12 may, in some instances, be implanted between the left posterior axillary line and the left anterior axillary line of the patient. Housing 24 of IMD 12 may, however, be implanted at other extrathoracic locations on the patient, implanted in an intrathoracic location, or not implanted at all in the case of an external pacemaker.

In the example of FIG. 1, lead 14 includes electrode 22A and electrode 22B (collectively referred to as one or more electrodes 22, electrodes 22, or first subset of electrodes 22) configured to deliver electrical therapy to heart 19 of patient 13 or sense cardiac activity of heart 19. In some examples, electrodes 22 are configured to deliver cardiac pacing, such as bradycardia pacing, asystole pacing, anti-tachycardia therapy (ATP) pacing, post-shock pacing, or cardiac resynchronization therapy (CRT) pacing. In some examples, electrodes 22 are additionally or alternatively configured to sense cardiac activity (e.g., electrical activity of heart 19). In some examples, electrodes 22 are additionally or alternatively configured to deliver an anti-tachyarrhythmia, e.g., cardioversion/defibrillation, shock to heart 19 of patient 13. While shown as two electrodes in the example of FIG. 1, electrodes 22 can include at least one or more electrodes.

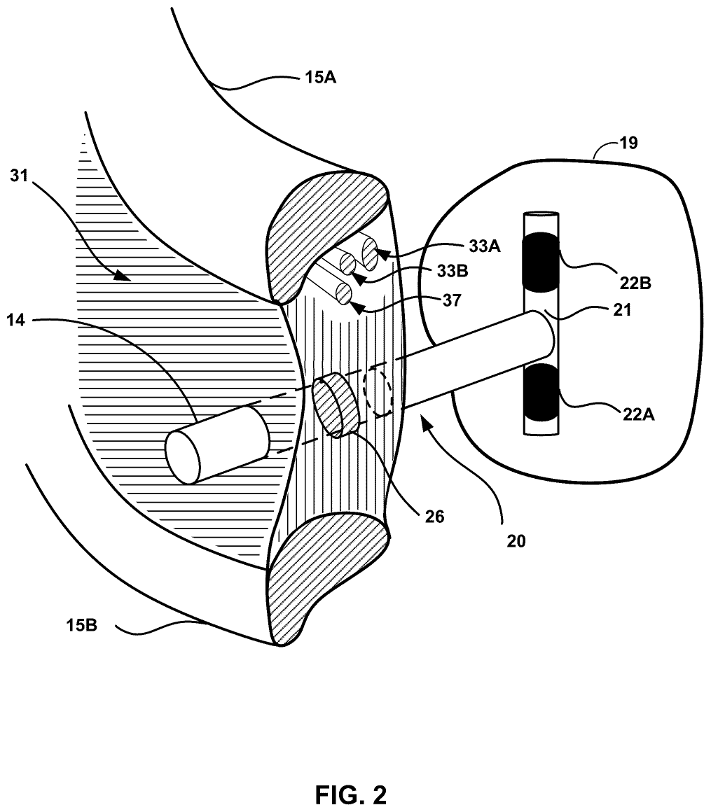

In some examples, electrodes 22 are carried by distal portion 20 of lead 14. Electrodes 22 may be disposed on lead 14 or embedded within the lead body 16 of lead 14. Examples of electrodes 22 include segmented electrodes, circumferential electrodes, ring electrodes, ribbon electrodes, coil electrodes, paddle electrodes, hemispherical electrodes, button electrodes, directional electrodes, mesh electrodes, defibrillation electrodes, etc., and may be positioned at any position along lead 14. While the example of FIGS. 1-2 illustrate electrodes 22 positioned anterior to heart 19, electrodes 22 may be positioned on a side (e.g., right or left), posteriorly, superiorly, or inferiorly relative to heart 19. Electrodes 22 (and lead 14) may be positioned according to a type of therapy to be delivered to heart 19 via electrodes 22. In some examples, electrodes 22 are positioned relative to a given chamber (e.g., right atrium, left atrium, right ventricle, or left ventricle) or area of heart 19 according to the type of therapy to be delivered to heart 19 via electrodes 22.

While the example of FIG. 1 shows a single lead 14 with electrodes 22, system 10 may include any number of leads with electrodes. For examples, multiple leads 14 may be used to position electrodes 22 at various locations, including, but not limited to, locations both anterior and posterior of heart 19 of patient 13. As another example, multiple leads may extend through intercostal locations as well as substernal locations within patient 13. As another example, system 10 may include leads that branch or split (e.g., branch at a location distal of where lead body 16 connects to housing 24). As another example, some or all of electrodes 22 may additionally or alternatively be carried by or disposed on housing 24.

In the example of FIG. 1, lead 14 includes one or more electrodes 26 (collectively also referred to as electrodes 26 or second subset of electrodes 26) configured to deliver energy to tissue proximate ribcage 15 of the patient to reduce a sensation in one or more intercostal nerves. Because lead 14 is configured to remain implanted, electrodes 26 may be configured to remain implanted in patient 13 after lead 14 is navigated through patient 13 in examples where electrodes 26 are carried by lead 14. In some examples, electrodes 26 are configured to be oriented toward intercostal space 31 and/or away from heart 19. In some examples, electrodes 26 are configured to deliver at least one of peripheral nerve stimulation, electroporation energy, or another form of therapy anterior to heart 19 (e.g., to tissue proximate to ribs 15 of patient 13). In some examples, a therapy agent (e.g., a drug or other chemical) may be carried by lead 14 to reduce a sensation in one or more intercostal nerves. In some examples, lead 14 is configured to deliver the therapy agent to tissue proximate ribcage 15 of patient 13.

In some examples, electrodes 26 are configured to reduce a sensation (e.g., reduce pain, paresthesia, etc.) associated with navigation and placement of lead 14 (or another elongated structure) through intercostal space 31. When lead 14 is implanted in or through intercostal space 31 (e.g., via a delivery device, access tool, trocar, etc.), one or more intercostal nerves may be disturbed during the implant procedure, or may be disturbed by lead 14 and/or tissue (e.g., tissue experiencing inflammation) after lead 14 (or another elongated structure) is implanted in, through, or proximate to intercostal space 31. In some examples, lead 14 (or other leads) may not be implanted directly through intercostal space 31, but may nonetheless cause an unwanted disturbance to one or more intercostal nerves. The disturbance of the one or more intercostal nerves may cause patient 13 to experience a sensation (e.g., pain, paresthesia, etc.). Delivery of therapy via electrodes 26 positioned in intercostal space 31 and/or directed at one or more intercostal nerves may reduce or eliminate the sensation (e.g., pain, paresthesia, etc.) caused by lead 14. For example, delivery of continuous therapy or at one or more intervals to one or more intercostal nerves by electrodes 26 may reduce or eliminate the sensation (e.g., pain, paresthesia, etc.) in the one or more intercostal nerves. Electrodes 26 may be configured to desensitize one or more intercostal nerves. Because electrodes 26 is configured to remain implanted, electrodes 26 may reduce chronic sensations that otherwise may go untreated as compared to other acute treatments performed during or shortly after implantation of IMD 12 and lead 14, which may only treat acute sensations.

In some examples, electrodes 26 are configured to reduce a sensation (e.g., reduce pain, paresthesia, etc.) associated with electrical therapy from electrodes 22. In some examples, when electrodes 22 are delivering electrical therapy to heart 19, an unwanted sensation (e.g., pain, paresthesia, etc.) may be experienced in various location because of the electrical therapy directed to heart 19. For example, when electrodes 22 are delivering electrical therapy to heart 19, patient 13 may experience a sensation in one or more intercostal nerves. In some examples, the sensation in one or more intercostal nerves caused by delivery of electrical therapy to heart 19 may be painful. In some examples, the sensation in the intercostal nerves caused by delivery of electrical therapy to heart 19 may not be painful but may be otherwise unpleasant or unwanted. Delivery of therapy via electrodes 26 positioned in intercostal space 31 and/or directed at one or more intercostal nerves may reduce or eliminate the sensation (e.g., pain, paresthesia, etc.) caused by delivery of electrical therapy to heart 19. In some examples, electrodes 26 may be configured to deliver therapy in intercostal space 31 and/or directed at one or more intercostal nerves while electrodes 22 are delivering electrical therapy to heart 19 (e.g., at the same time, or around the same time frame). In some examples, delivery of electrical therapy to heart 19 via electrodes 22 may trigger delivery of therapy via electrodes 26 directed at one or more intercostal nerves may reduce or eliminate the sensation (e.g., pain, paresthesia, etc.).

In some examples, electrodes 26 are configured to reduce or eliminate a sensation (e.g., reduce pain, paresthesia, etc.) associated with placement of housing 24 and/or stimulation proximate housing 24. In some examples, at least some of electrodes 26 are carried by housing 24. In some examples, at least some of electrodes 26 are configured to be positioned proximate housing 24 (e.g., via a separate lead). In examples where housing 24 includes a housing electrode, can electrode, or other therapy electrode, electrodes 26 may be configured to reduce or eliminate a sensation proximate housing 24.

In some examples, electrodes 26 are configured to sense a patient parameter. Electrodes 26 may be configured to sense electrical activity of heart 19. IMD 12 may be configured to sense a heart rate or another parameter from heart 19 via electrodes 26, e.g., based on the electrical activity of heart 19. IMD 12 may be configured to sense, e.g., via electrodes 26, an evoked compound action potential (ECAP) from surrounding tissue. As discussed below, IMD 12 may include one or more additional sensors (e.g., accelerometers, piezoelectric sensors, acoustic sensors, microphones, etc.). The one or more additional sensors may be configured to sense a patient parameter. The one or more additional sensors may be disposed on, incorporate into, or otherwise carried by lead 14, housing 25 of IMD 12, or another suitable portion of system 10. In some examples, a sensed parameter may be associated with a severity or level of sensation experienced by patient 13, such that the sensed parameter is used to modulate the therapy provided by electrodes 26. For example, a change in a level of a sensed parameter (e.g., electrical activity of heart 19, heart sounds, ECAP, etc.) may trigger IMD 12 to adjust a level, frequency, or duration of therapy provided to tissue adjacent ribcage 15 of patient 13. The change in a level of a sensed parameter may be indicative of electrical therapy directed at heart 19. In some examples, IMD 12 may automatically change therapy based on the sensed patient parameter to reduce or eliminate a sensation in one or more intercostal nerves.

Although the first subset of electrodes 22 and second subset of electrodes 26 may be configured as described above, in some examples, one or more electrodes from first subset of electrodes 22 and second subset of electrodes 26 may be configured to perform functions of both sets of electrodes. For examples, first subset of electrodes 22 and second subset of electrodes 26 may share at least one common electrode. The at least one common electrode between first subset of electrodes 22 and second subset of electrodes 26 may be configured to deliver energy to tissue proximate ribcage 15 of the patient to reduce a sensation in one or more intercostal nerves and configured to deliver electrical therapy to heart 19 of patient 13. As another example, the at least one common electrode between first subset of electrodes 22 and second subset of electrodes 26 may be configured to deliver energy to tissue proximate ribcage 15 of the patient to reduce a sensation in one or more intercostal nerves and configured to sense cardiac activity of heart 19 of patient 13. As another example, the at least one common electrode between first subset of electrodes 22 and second subset of electrodes 26 may be configured to deliver energy to tissue proximate ribcage 15 of the patient to reduce a sensation in one or more intercostal nerves, configured to deliver electrical therapy to heart 19 of patient 13, and configured to sense cardiac activity of heart 19 of patient 13. As another example, the at least one common electrode between first subset of electrodes 22 and second subset of electrodes 26 may be configured to sense a patient parameter (e.g., electrical activity of heart 19, ECAP, etc.).

While the example of FIG. 1 shows a single electrode 26 disposed on distal portion 20 of lead 14 and within intercostal space 31, system 10 may include any number electrodes 26 (e.g., one, two, three, four, or more) disposed on any suitable structure. For example, multiple leads 14 may be used to position electrodes 26 at various locations, including, but not limited to, locations both anterior and posterior of heart 19 of patient 13. In some examples, one or more leads 14 may position electrodes 26 in one or more locations (e.g., within intercostal space 31, adjacent intercostal space 31, adjacent housing 24, etc.). In some examples, one or more electrodes 26 may be configured to be positioned proximate the xiphoid process. In some examples, one or more electrodes 26 may be configured to be positioned proximate housing 24. As another example, some or all of electrodes 26 may additionally or alternatively be carried by or disposed on housing 24.

FIG. 2 is a conceptual diagram illustrating a perspective view of system 10 from FIG. 1, including distal portion 20 of lead 14 positioned through intercostal space 31. In the example of FIG. 2, distal portion 20 of lead 14 is positioned through intercostal space 31, which may be between a first rib 15A (e.g., a superior rib 15A) and a second rib 15B (e.g., an inferior rib 15B) (collectively ribs 15, as discussed in connection with FIG. 1). As discussed previously, lead 14 is configured to extend from an access point of patient 13 through intercostal space 31. While electrode 26 is shown in the intercostal space 31 directly between first rib 15A and second rib 15B, electrode 26 may be positioned anterior or posterior relative to the centerline of the space between first rib 15A and second rib 15B. In some examples, electrode 26 spans across intercostal space 31 between first rib 15A and second rib 15B. In some examples, lead 14 rests on rib 15B (e.g., where rib 15B is inferior to lead 14 and rib 15A is superior to lead 14).

Distal portion 20 of lead 14 may be oriented relative to an intercostal vein 33A, an intercostal artery 33B, and an intercostal nerve 37. For example, distal portion 20 of lead 14 may be oriented to position electrode 26 closer to intercostal nerve 37 and further away from intercostal vein 33A and intercostal artery 33B (collectively, intercostal vascular bundle 33). Distal portion 20 of lead 14 may be oriented to position electrode 26 to direct delivery of an electric field via electrode 26 toward intercostal nerve 37 (e.g., upwardly) and/or not direct delivery of an electric field toward intercostal vascular bundle 33. In some examples, as described below, distal portion 20 of lead 14 may include one or more shields configured to impede delivery of an electric field via electrode 26 to intercostal vein 33A, intercostal artery 33B, or collectively to intercostal vascular bundle 33. In some examples, as described below, distal portion 20 of lead 14 may include one or more shields configured to impede delivery of an electric field via electrode 26 toward heart 19.

Electrode 26 may be configured to deliver energy proximate rib 15A and rib 15B to reduce a sensation in intercostal nerve 37. In some examples, electrode 26 may include one or more of a circumferential electrode, a ring electrode, a segmented electrode, a coil electrode, a paddle electrode, an electrode array, another suitable type of electrode, or any combination thereof. In some examples, electrode 26 is configured to deliver energy to other tissue in intercostal space 31 (e.g., muscle tissue or other connective tissue). In some examples, electrode 26 is configured to deliver energy to reduce a sensation in nerves besides one or more intercostal nerves 37, including nerves in muscle or other connective tissue proximate ribs 15 or in the thoracic cavity.

As shown in the example of FIG. 2, lead 14 may include an electrode carrying portion 21. Electrode carrying portion 21 may be configured to carry at least electrodes 22. Electrode carrying portion 21 may be coupled to lead 14. Electrode carrying portion 21 may branch from (e.g., in a direction perpendicular to) lead 14. Electrode carrying portion 21 may be configured to orient electrodes 22 relative to heart 19. In some examples, electrode carrying portion 21 may orient electrodes 22 to face toward heart 19. First electrode 22A may be positioned at a first end of electrode carrying portion 21, and second electrode 22B may be positioned at a second end of electrode carrying portion 21. Electrode carrying portion 21 may include a different material than lead 14, or electrode carrying portion 21 may include the same material as lead 14. Electrode carrying portion 21 may be integrally formed with lead 14. Electrode carrying portion 21 may be separately formed from lead 14 and coupled to lead 14.

While the example of FIG. 2 shows a single electrode 26 disposed on distal portion 20 of lead 14 and within intercostal space 31, any number of electrodes 26 (e.g., one, two, three, four, or more) may be disposed on any suitable structure and oriented in any suitable configuration. For example, multiple leads 14 may be used to position electrodes 26 at various locations, including, but not limited to, locations both anterior and posterior of heart 19 of patient 13. In some examples, one or more leads 14 may position electrodes 26 in one or more locations (e.g., within intercostal space 31, adjacent intercostal space 31, etc.). In some examples, one or more electrodes 26 may be disposed on electrode carrying portion 21. In some examples, electrodes 26 are configured to touch ribs 15. In some examples, electrodes 26 are configured to face ribs 15. For example, when electrodes 26 are positioned outside of ribcage 15, electrodes 26 may face inward towards ribs 15. In some examples, when electrodes 26 are positioned inside of ribcage 15, electrodes 26 may face outwards towards ribs 15.

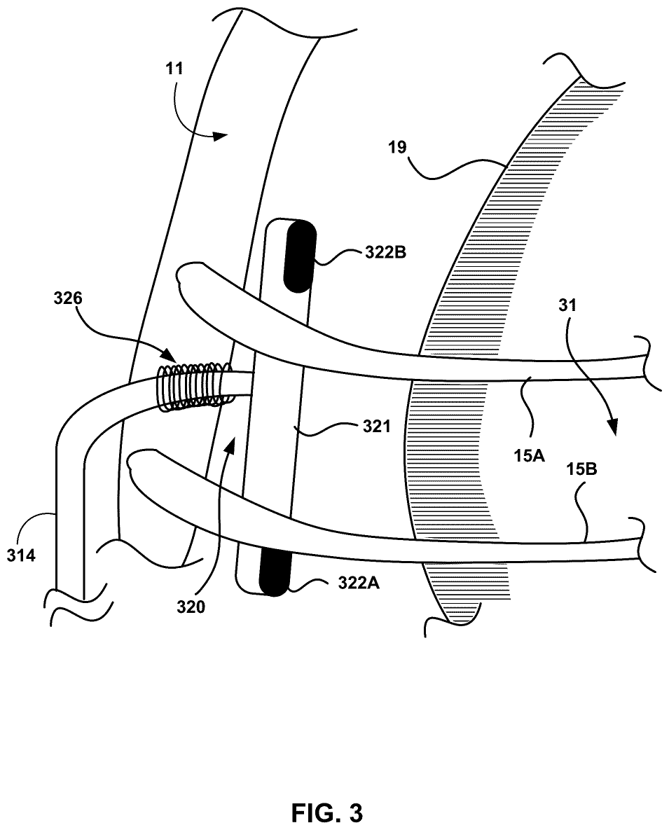

FIG. 3 is a conceptual diagram illustrating a side view of a distal portion 320 of a lead 314, which may be an example of distal portion 20 of lead 14 as illustrated in FIGS. 1-2. In the example of FIG. 3, lead 314 includes a first subset of electrodes 322 (shown individually as electrode 322A and electrode 322B) and a second subset of electrodes 326 (shown individually as electrode 326). Electrodes 322 may be carried by a distal portion 320 of lead 314. In some examples, electrodes 322 are carried on an electrode carrying portion 321, with electrode carrying portion 321 coupled to or extending from lead 314. In the example of FIG. 3, electrode 326 is disposed on distal portion 320 of lead 314, but not disposed on electrode carrying portion 321.

Electrodes 322 may be configured to deliver electrical therapy to heart 19 of the patient or sense cardiac activity and may otherwise be configured according to the description of electrodes 22 in the examples of FIGS. 1-2. In some examples, electrodes 322 are configured to deliver cardiac pacing, such as bradycardia pacing, asystole pacing, anti-tachycardia therapy (ATP) pacing, post-shock pacing, or cardiac resynchronization therapy (CRT) pacing. In some examples, electrodes 322 are additionally or alternatively configured to sense cardiac activity (e.g., electrical activity of heart 19). In some examples, electrodes 322 are additionally or alternatively configured to deliver an anti-tachyarrhythmia, e.g., cardioversion/defibrillation, shock to heart 19.

In the example of FIG. 3, electrode 326 is a coil electrode disposed on distal portion 320 of lead 314 and positioned proximal of electrodes 322 on lead 314. Electrode 326 may be configured according to the description of electrode 26 in the examples of FIGS. 1-2 except as described herein and may be used in addition to or instead of other electrodes configured for reducing a sensation in one or more intercostal nerves. Electrode 326 may be positioned in (e.g., through) intercostal space 31. Electrode 326 may be positioned between a first rib 15A (e.g., a superior rib 15A) and a second rib 15B (e.g., an inferior rib 15B). Electrode 326 may be configured to direct energy to one or more intercostal nerves in intercostal space 31.

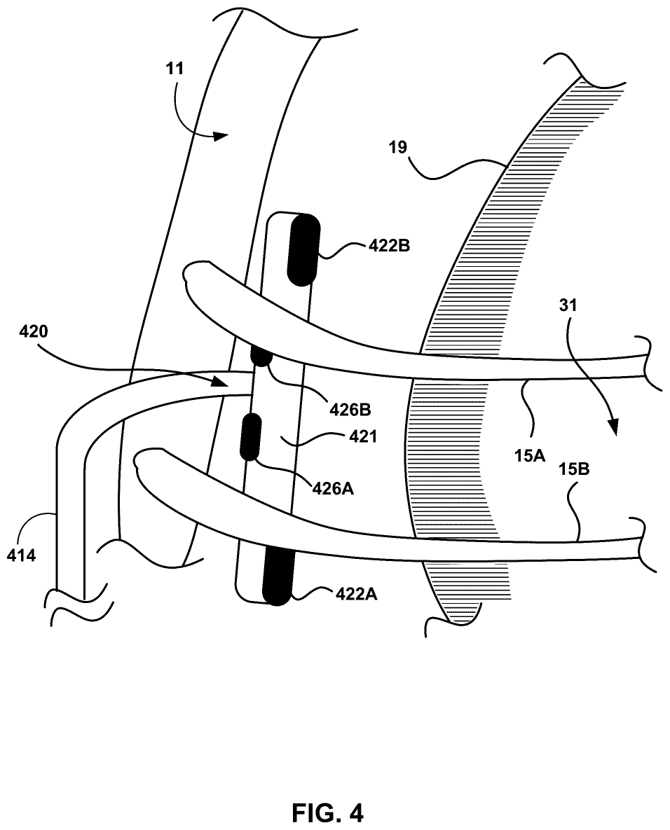

FIG. 4 is a conceptual diagram illustrating a side view of a distal portion 420 of a lead 414, which may be an example of distal portion 20 of lead 14 as illustrated in FIGS. 1-2. In the example of FIG. 4, lead 414 includes a first subset of electrodes 422 (shown individually as electrode 422A and electrode 422B) and a second subset of electrodes 426 (shown individually as electrode 426A and electrode 426B). Electrodes 422 may be carried by distal portion 420 of lead 414. In some examples, electrodes 422 are carried on an electrode carrying portion 421, with electrode carrying portion 421 coupled to or extending from lead 414. In the example of FIG. 4, electrodes 426 are also disposed on electrode carrying portion 421.

Electrodes 422 may be configured to deliver electrical therapy to heart 19 of the patient or sense cardiac activity and may otherwise be configured according to the description of electrodes 22 in the examples of FIGS. 1-2. In some examples, electrodes 422 are configured to deliver cardiac pacing, such as bradycardia pacing, asystole pacing, anti-tachycardia therapy (ATP) pacing, post-shock pacing, or cardiac resynchronization therapy (CRT) pacing. In some examples, electrodes 422 are additionally or alternatively configured to sense cardiac activity (e.g., electrical activity of heart 19). In some examples, electrodes 422 are additionally or alternatively configured to deliver an anti-tachyarrhythmia, e.g., cardioversion/defibrillation, shock to heart 19.

In some examples, electrodes 426 are segmented electrodes, partial ring electrodes, paddle electrodes, and/or another suitable type of electrode disposed on electrode carrying portion 421 coupled to lead 414. Electrodes 426 may be disposed on a portion of electrode carrying portion 421 configured to face away from the heart. Electrodes 426 may be disposed on a portion of electrode carrying portion 421 configured to face toward rib 15A, rib 15B, and/or intercostal space 31. Electrode 426 may be configured according to the description of electrode 26 in the examples of FIGS. 1-2 except as described herein and may be used in addition to or instead of other electrodes configured for reducing a sensation in one or more intercostal nerves. Electrode 426 may be positioned adjacent to or near intercostal space 31, but not directly in or through intercostal space 31. In some examples, when electrode carrying portion 421 orients electrodes 422 toward heart 19, electrode carrying portion simultaneously orients electrodes 426 toward intercostal space 31 and/or one or more intercostal nerves. Electrode 426 may be configured to direct energy to one or more intercostal nerves in intercostal space 31.

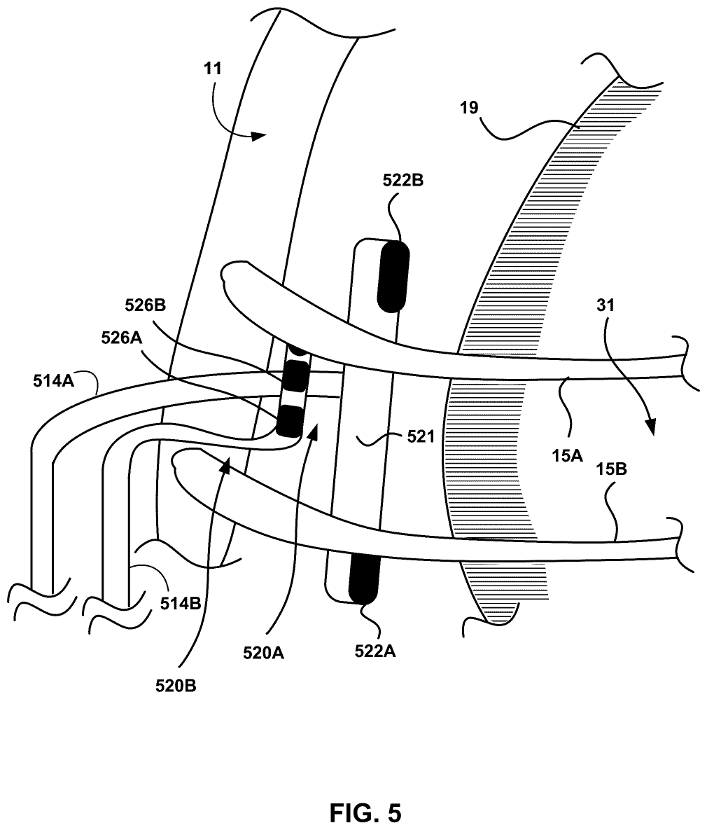

FIG. 5 is a conceptual diagram illustrating a side view of an example system with multiple elongated bodies, which may be used an addition to or instead of the leads illustrated in FIGS. 1-4. In the example of FIG. 5, a first subset of electrodes 522 (shown individually as electrode 522A and electrode 522B) are carried by a distal portion 520A of a first lead 514A, which may be an elongated body. In some examples, electrodes 522 are carried on an electrode carrying portion 521 coupled to or extending from lead 514A. A second set of electrodes 526 (shown individually as electrode 526A and electrode 526B) are carried by a distal portion 520B of a second lead 514B, which may be a second elongated body. In some examples, lead 514B branches from lead 514A at a location along the length of the body of lead 514A. In some examples, lead 514B is a fold diverging from lead 514A at a location along the length of the body of lead 514A. In some examples, lead 514B is separate from lead 514A, but connected to a common implantable medical device with lead 514A (e.g., IMD 12, as shown in FIG. 1). In some examples, lead 514B is separate from lead 514A and also connected to a different implantable medical device or other generator as lead 514A. In some examples, lead 514B is configured to be positioned proximate a housing of an implantable medical device (e.g., housing 24 of IMD 12, as shown in FIG. 1).

Electrodes 522 may be configured to deliver electrical therapy to heart 19 of the patient or sense cardiac activity and may otherwise be configured according to the description of electrodes 22 in the examples of FIGS. 1-2. In some examples, electrodes 522 are configured to deliver cardiac pacing, such as bradycardia pacing, asystole pacing, anti-tachycardia therapy (ATP) pacing, post-shock pacing, or cardiac resynchronization therapy (CRT) pacing. In some examples, electrodes 522 are additionally or alternatively configured to sense cardiac activity (e.g., electrical activity of heart 19). In some examples, electrodes 522 are additionally or alternatively configured to deliver an anti-tachyarrhythmia, e.g., cardioversion/defibrillation, shock to heart 19.

Electrodes 526 may be configured according to the description of electrode 26 in the examples of FIGS. 1-2 except as described herein and may be used in addition to or instead of other electrodes configured for reducing a sensation in one or more intercostal nerves. As shown in the example of FIG. 5, lead 514B extends through intercostal space 31 to position electrodes 526 adjacent to rib 15A, rib 15B, and/or intercostal space 31 to reduce a sensation in one or more intercostal nerves. In some examples, lead 514B extends completely through intercostal space 31 so that a distal portion 520B of lead 514B is positioned adjacent electrode carrying portion 521 of lead 514A. Electrodes 526 may face away from heart 19 and toward intercostal space 31. Electrodes 526 may face in a direction opposite of electrodes 522.

Lead 514B may be positioned and/or oriented to provide effective therapy to one or more intercostal nerves. For example, lead 514B may be positioned such that distal portion 520B of lead 514, including electrodes 526, are positioned in intercostal space 31 (e.g., electrodes 526 are positioned to deliver therapy between a first rib 15A and a second rib 15B and electrodes 526 are oriented to direct an electric field upward toward one or more intervascular nerves). In some examples, lead 514B is positioned such that distal portion 520B of lead 514B, including electrodes 526, are positioned outside of (e.g., anterior to) ribs 15. When lead 514B is positioned outside of ribs 15, electrodes 526 face inward toward intercostal space 31. Lead 514B may extend to an intercostal location adjacent where lead 514A extends through intercostal space 31.

While the example of FIG. 5 illustrates lead 514B extending through intercostal space 31, lead 514B may extend in other ways to position electrodes 526 for delivery of energy to one or more intercostal nerves. In some examples, lead 514B extends under the ribcage and sternum 11 (e.g., substernally) to position electrodes 526 for delivery of energy to one or more intercostal nerves. In some examples, lead 514B extends on the outside of the ribcage such that electrodes 526 face inward toward intercostal space 31.

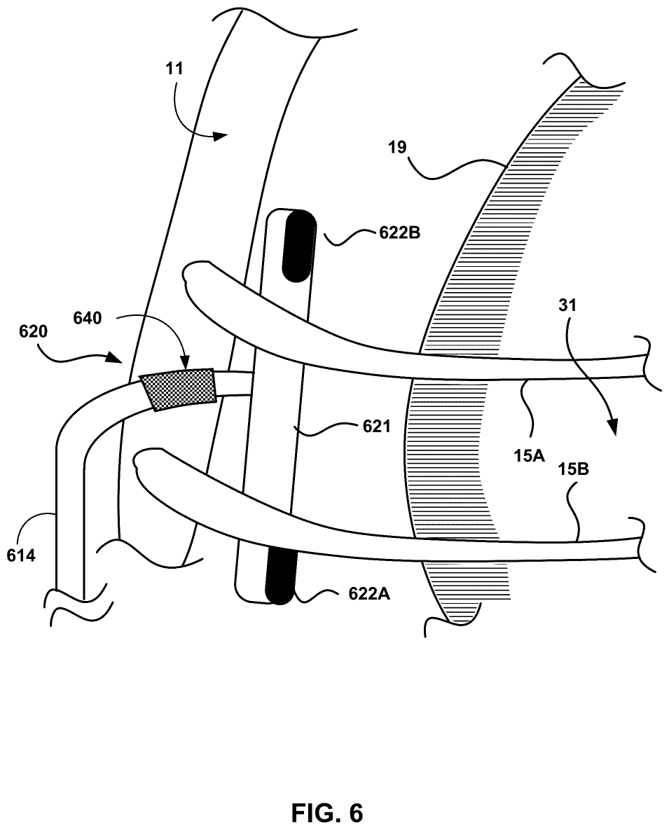

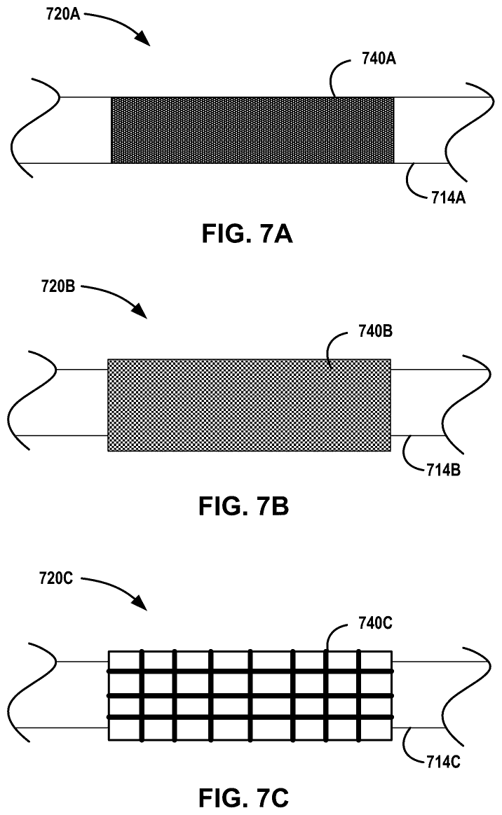

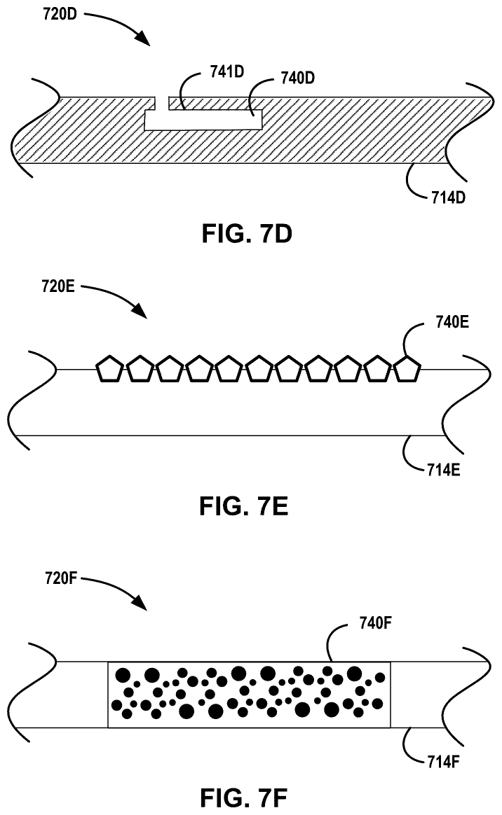

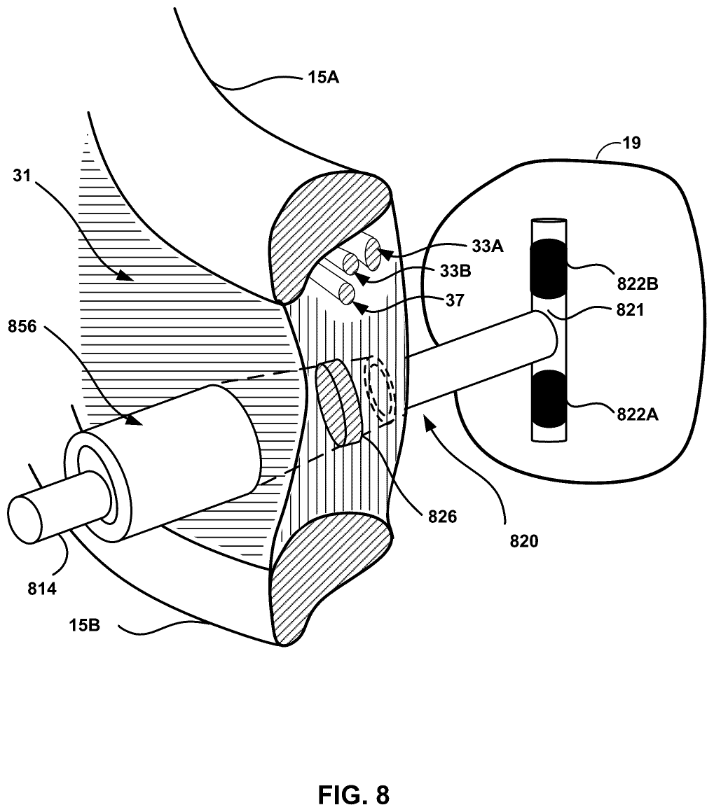

FIG. 6 is a conceptual diagram illustrating a side of view of a distal portion 620 of a lead 614, which may be an example of distal portion 20 of lead 14 as illustrated in FIGS. 1-2. In the example of FIG. 6, lead 614 includes a first subset of electrodes 622 (shown individually as electrode 622A and electrode 622B). Electrodes 622 are carried by a distal portion 620 of lead 614. In some examples, electrodes 622 are carried on an electrode carrying portion 621 coupled to or extending from lead 614. In the example of FIG. 6, a distal portion 620 of lead 614 includes a therapy agent 640 configured to reduce inflammation, reduce a sensation in one or more intercostal nerves, kill or reduce the growth of microbes, and/or produce another therapeutical effect. Therapy agent 640 may include at least one of an anti-inflammatory agent configured to inhibit inflammation, an analgesic agent configured to relieve pain or reduce sensation, a neurotoxin, or an antimicrobial agent configured to kill or slow growth of microbes. As described further in the examples of FIGS. 7A-7F, therapy agent 640 may be incorporated into, carried by, disposed on, or otherwise delivered by distal portion 620 of lead 614. In some examples, therapy agent 640 may additionally or alternatively be incorporated into, carried by, disposed on, or otherwise delivered by an electrode carrying portion 621. Therapy agent 640 may be used in connection with any of the previous examples, including examples in which electrodes are configured to reduce a sensation in one or more intercostal nerves. Lead 614 may be configured to deliver therapy agent 640 to tissue proximate ribcage 15 or into intercostal space 31.

As shown in FIG. 6, therapy agent 640 may be included along a portion of lead 614 for delivery of the therapy agent 640 to intercostal space 31. In some examples, therapy agent 640 is included along a portion of lead 614 positioned in or through intercostal space 31. In some examples, therapy agent 640 is additionally or alternatively included along a portion of lead 614 positioned outside of intercostal space 31. Therapy agent 640 may be included along multiple portions of lead 614 (e.g., both portions of lead 614 in or through intercostal space 31 and portions of lead 614 outside of intercostal space 31). Therapy agent 640 may also be included on electrode carrying portion 621. In some examples, therapy agent 640 is delivered into intercostal space 31. In some examples, therapy agent 640 is delivered adjacent to intercostal space 31.

In some examples, therapy agent 640 includes one or more molecules or compounds to be delivered to tissue of a patient to produce one or more therapeutical effects (herein after referred to as an “active molecule or compound”). For example, therapy agent 640 may include molecules or compounds configured to inhibit inflammation of tissue. Therapy agent 640 may additionally or alternatively include molecules or compounds configured to reduce pain or other sensation in a patient. Therapy agent 640 may additionally or alternatively include one or more antimicrobial molecules or compounds. In some examples, therapy agent 640 includes one or more analgesic compounds including, but not limited to lidocaine, bupivacaine, and/or gabapentin. In some examples, therapy agent 640 includes one or more nonsteroidal anti-inflammatory drugs (NSAIDs). In some examples, therapy agent 640 includes one or more steroids. In some examples, therapy agent 640 includes botulinum neurotoxin. As discussed in connection with FIGS. 7A-7F, therapy agent 640 may include or otherwise be incorporate into materials or structures configured to aid in the delivery of therapy agent 640 to intercostal space 31. Therapy agent 640 may further include a combination of different molecules or compounds configured to produce different therapeutical effects.

FIGS. 7A-7F are conceptual diagrams illustrating a lead 714 (shown individually as lead 714A, lead 714B, lead 714C, lead 714D, lead 714E, and lead 714F in FIGS. 7A-7F, respectively) including a therapy agent 740 (shown individually as therapy agent 740A, therapy agent 740B, therapy agent 740C, therapy agent 740D, therapy agent 740E, and therapy agent 740F in FIGS. 7A-7F, respectively). Lead 714 includes a distal portion 720 (shown individually as distal portion 720A, distal portion 720B, distal portion 720C, distal portion 720D, distal portion 720E, and distal portion 720F in FIGS. 7A-7F, respectively) where therapy agent 740 may be located. Lead 714 may be an example of lead 14 of FIGS. 1-2 and/or lead 614 of FIG. 6, except as described herein. Therapy agent 740A, 740B, 740C, 740D, 740E, and 740F may be examples of therapy agent 640 of FIG. 6, except as described herein. As described below, therapy agent 740 may be configured to release an active molecule or compound configured to produce one or more therapeutic effects. The release of the active molecule or compound may be time-controlled such that a therapeutically effective dose of the active molecule or compound is released over a period of time. For example, therapy agent 740 may be configured for short-term release of a drug, chronic (e.g., long term) drug release (e.g., elution), or a combination of both short-term and long-term release.

FIG. 7A is a conceptual diagram illustrating a portion of a lead 714A including a therapy agent 740A. In the example of FIG. 7A, lead 714A includes therapy agent 740A as a coating, overlay, or film disposed on an outer surface of the lead body of lead 714A at (or proximate to) a distal portion 720A of lead 714A. In some examples, the coating comprises therapy agent 740A. Therapy agent 740A as a coating, overlay, or film may include one or more materials configured to aid with securing the coating, overlay, or film on lead 714A. In some examples, therapy agent 740A includes one or more additional compounds for securing therapy agent 740A to lead 714A along with the active molecule or compound. Therapy agent 740A may be applied to lead 714A as a spray coating, a dip coating, or other method of applying a coating, overlay, or film. Therapy agent 740A may be configured to release the active molecule or compound to tissue of the patient over a period of time (e.g., depending on the composition, thickness, or application of therapy agent 740A to lead 714A).

FIG. 7B is a conceptual diagram illustrating a portion of a lead 714B including a therapy agent 740B. In the example of FIG. 7B, lead 714B includes therapy agent 740B as a sleeve or wrap disposed over an outer surface of the lead body of lead 714B at (or proximate to) a distal portion 720B of lead 714B. In some examples, the sleeve or wrap comprises therapy agent 740B. Therapy agent 740B may include one or more of a polymer or other material configured to act as a substrate that may be disposed around lead body of lead 714B. In some examples, therapy agent 740B as a sleeve or wrap includes a resorbable material, including but not limited to poly(lactic-co-glycolic acid) (PLGA), hyaluronic acid/gel, or another suitable material. In some examples, the resorbable material includes therapy agent 740B. The resorbable material may be cross-linked with one or more polymers. In some examples, the materials used for the sleeve or wrap are configured to provide cushion to surrounding tissue in the intercostal space.

FIG. 7C is a conceptual diagram illustrating a portion of lead 714C including a therapy agent 740C. In the example of FIG. 7C, lead 714C includes therapy agent 740C as a mesh disposed over an outer surface of the lead body of lead 714C at (or proximate to) a distal portion 720C of lead 714C. In some examples, the mesh comprises therapy agent 740C. In some examples, therapy agent 740C includes a multi-filament knitted mesh configured to be disposed over the lead body of lead 714C (e.g., the mesh of Medtronic TYRX Absorbable Antibacterial Envelope). Therapy agent 740C including the mesh may include the resorbable materials noted above. In some examples, the resorbable material comprises therapy agent 740C.

FIG. 7D is a conceptual diagram illustrating a cross-section view of portion of a lead 714D including a therapy agent 740D, the cross-section taken along a direction parallel to a major longitudinal axis of lead 714D. In the example of FIG. 7D, lead 714D includes therapy agent 740D disposed in a reservoir 741D within the lead body of lead 714D at a distal portion 720D of lead 714D. Reservoir 741D may be shaped, size, or otherwise configured to hold therapy agent 740D. In some examples, therapy agent 740D is eluted from lead 714D via reservoir 741D. Reservoir 741D may be configured to release therapy agent 740D at a controlled rate. In some examples, lead 714D includes more than one reservoir configured to hold therapy agent 740D. The body of lead 714D may define reservoir 741D.

FIG. 7E is a conceptual diagram illustrating a portion of lead 714E including a therapy agent 740E. In the example of FIG. 7E, lead 714E includes therapy agent 740E in a crystalline structure disposed over an outer surface of the lead body of lead 714E at (or proximate to) a distal portion 720E of lead 714E. In some examples, therapy agent 740E includes a crystalline structure configured to be forced into tissue (e.g., tissue in the intercostal space between ribs). In some examples, therapy agent 740E including a crystalline structure may be configured to deployed (e.g., via an expandable balloon, stent, or other deployable structure) to be force into tissue of a patient.

FIG. 7F is a conceptual diagram illustrating a portion of lead 714F including a therapy agent 740F. In the example of FIG. 7F, lead 714F includes therapy agent 740F incorporated into the lead body of lead 714F at (or proximate to) a distal portion 720F of lead 714F. For example, therapy agent 740F may be incorporated into the body of lead 714F, which may include a polymer matrix. The polymer matrix of the body of lead 714F may be configured to release (e.g., elute) therapy agent 740F. Therapy agent 740F may be incorporated into a portion (e.g., an outer layer) of the lead body of lead 714F.

While the examples of FIGS. 7A-7F are described individually, the examples may be used in any combination without limitation. For example, lead 714 may include therapy agent 740 both as a coating (e.g., as depicted in FIG. 7A) as well as being incorporated into the lead body of lead 714 (e.g., as depicted in FIG. 7E and FIG. 7F).

FIG. 8 is a conceptual diagram illustrating a perspective view of system including a delivery device 856. A delivery device 856 may be configured to assist in navigating a lead 814 through intercostal space 31. In some examples, delivery device 856 includes a trocar or other tool configured to deliver medical devices through intercostal space 31. Delivery device 856 may be configured to be inserted into intercostal space 31 during an implant procedure, and subsequently removed after the implant procedure. Lead 814, including electrode 822A and electrode 822B (collectively referred to as electrodes 822) disposed on an electrode carrying portion 821 of lead 814 may be configured to be inserted through delivery device 856. As discussed in connection with earlier examples, electrodes 822 may be disposed on a distal portion 820 of lead 814 and configured to deliver electrical therapy to a heart 19.

As shown in the example of FIG. 8, delivery device 856 includes at least one electrode 826 configured to deliver energy to tissue proximate the ribcage 15 (e.g., proximate rib 15A and/or rib 15B), such as including intercostal space 31 to reduce a sensation in one or more intercostal nerves. Electrode 826 may be configured according to the description of electrodes 26 in the examples of FIGS. 1-2 except as described herein. In some examples, electrode 826 provides energy to intercostal tissue during an implant procedure when a lead 814 is navigated through intercostal space 31 to an implant location. In this way, electrode 826 may reduce a sensation associated with placement or navigation of lead 814 during an implant procedure. Electrode 826 may also produce chronic sensation reduction through desensitization of nerves. Because delivery device 856 is removed after the implant procedure, electrode 826 may also be configured to be removed after the implant procedure (e.g., not remain implanted after lead 814 has been implanted). Electrode 826 may be disposed around the circumference of delivery device 856. In some examples, electrode 826 is configured to direct electrical energy upward to an intercostal nerve 37 during an implant procedure. Delivery device 856 may include one or more therapy agents (as described above). For example, a therapy agent may be disposed on or carried by a surface of delivery device 856.

In some examples, delivery device 856 is configured to cauterize tissue during the implant procedure. Delivery device 856 may provide energy (e.g., thermal, radiofrequency, etc.) to tissue in intercostal space 31 to cauterize tissue. The cauterized tissue may be less susceptible to infection, inflammation, or sensation.

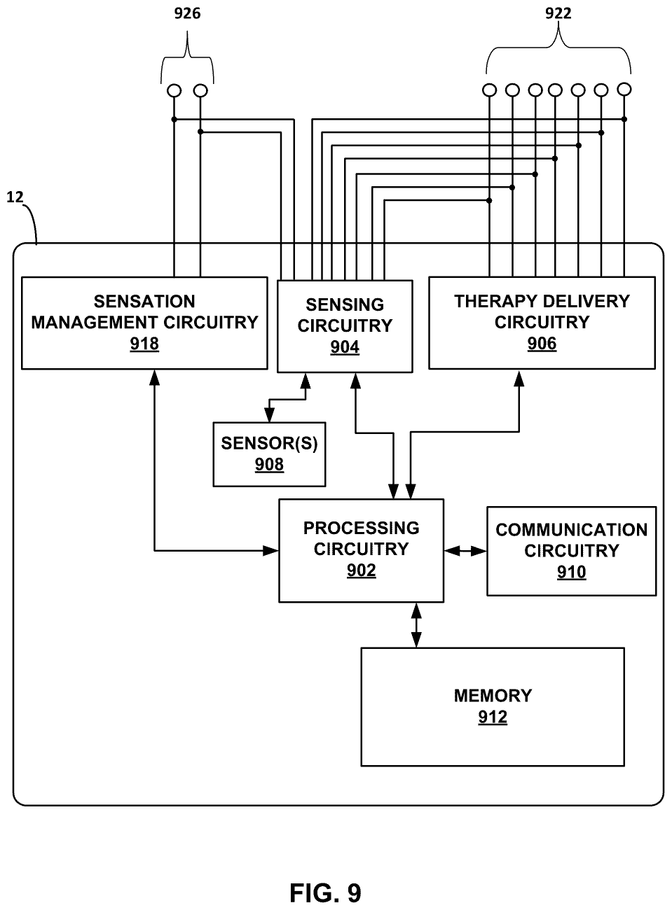

FIG. 9 is a functional block diagram of an example configuration of electronic components and other components of IMD 12. IMD 12 includes processing circuitry 902, sensing circuitry 904, therapy delivery circuitry 906, sensors 908, communication circuitry 910, memory 912, and sensation management circuitry 918. In some examples, IMD 12 may include more or fewer components. The described circuitry and other components may be implemented together on a common hardware component or separately as discrete but interoperable hardware or software components. Depiction of different features is intended to highlight different functional aspects and does not necessarily imply that such circuitry and other components must be realized by separate hardware or software components. Rather, functionality associated with one or more circuitries and components may be performed by separate hardware or software components, or integrated within common or separate hardware or software components.

Sensing circuitry 904 may be electrically coupled to some or all of electrodes 922 which may correspond to any of the defibrillation, pace/sense, and housing electrodes described herein (e.g., the first subset of the plurality of electrodes described above). Sensing circuitry 904 is configured to obtain signals sensed via one or more combinations of electrodes 922 and process the obtained signals. Similarly, sensing circuitry 904 may additionally or alternatively be coupled to some or all of electrodes 926, which may correspond to any of the electrodes configured for sensation management electrodes described herein (e.g., the second subset of the plurality of electrodes described above). Sensing circuitry 904 is configured to obtain signals sensed via one or more combinations of electrodes 926 and process the obtained signals.

The components of sensing circuitry 904 may be analog components, digital components or a combination thereof. Sensing circuitry 904 may, for example, include one or more sense amplifiers, filters, rectifiers, threshold detectors, analog-to-digital converters (ADCs) or the like. Sensing circuitry 904 may convert the sensed signals to digital form and provide the digital signals to processing circuitry 902 for processing or analysis. For example, sensing circuitry 904 may amplify signals from the sensing electrodes and convert the amplified signals to multi-bit digital signals by an ADC. Sensing circuitry 904 may also compare processed signals to a threshold to detect the existence of atrial or ventricular depolarizations (e.g., P- or R-waves) and indicate the existence of the atrial depolarization (e.g., P-waves) or ventricular depolarizations (e.g., R-waves) to processing circuitry 902. Sensing circuitry 904 may compare an ECAP signal to a threshold. As shown in FIG. 9, IMD 12 may additionally include one or more sensors 908, such as one or more accelerometers, which may be configured to provide signals indicative of other patient parameters, such as activity or posture, to processing circuitry 902. One or more sensors 908 may include one or more piezoelectric sensors, acoustic sensors, microphones, etc. configured to provide one or more signals indicative of one or more patient parameters. The one or more sensors 908 may be configured to sense heart sounds from heart 19 of patient 13.

Processing circuitry 902 may process the signals from sensing circuitry 904 to monitor electrical activity of heart 19 of patient 13. Processing circuitry 902 may store signals obtained by sensing circuitry 904 as well as any generated electrogram (EGM) waveforms, marker channel data or other data derived based on the sensed signals in memory 912. Processing circuitry 902 may analyze the EGM waveforms and/or marker channel data to detect arrhythmias (e.g., bradycardia or tachycardia). In response to detecting the cardiac event, processing circuitry 902 may control therapy delivery circuitry 906 to deliver the desired therapy to treat the cardiac event, e.g., defibrillation shock, cardioversion shock, anti-tachycardia pacing (ATP), post shock pacing, bradycardia pacing, or CRT.

Therapy delivery circuitry 906 is configured to generate and deliver electrical therapy to a heart. Therapy delivery circuitry 906 may include one or more pulse generators, capacitors, and/or other components capable of generating and/or storing energy to deliver as pacing therapy, defibrillation therapy, cardioversion therapy, CRT, other therapy, or a combination of therapies. Therapy delivery circuitry 906 may be configured to generate and deliver pacing pulses with magnitudes and timings specified by processing circuitry 902. For delivery of CRT, for example, processing circuitry 902 may control therapy delivery circuitry 906 to deliver pacing pulses according to one or more atrioventricular (AV) intervals from an intrinsic or paced atrial event, and/or one or more interventricular (VV) intervals from an intrinsic or paced ventricular event. The values of such intervals may be programmable by a user and/or variable based on physiological parameters sensed, e.g., via one or more sensors 908.

In some instances, therapy delivery circuitry 906 may include a first set of components configured to provide pacing therapy and a second set of components configured to provide defibrillation therapy. In some instances, therapy delivery circuitry 906 may utilize the same set of components to provide both pacing and defibrillation therapy. In still other instances, therapy delivery circuitry 906 may share some of the defibrillation and pacing therapy components while using other components solely for defibrillation or pacing. Processing circuitry 902 may control therapy delivery circuitry 906 to deliver the generated therapy to heart 19 via one or more combinations of electrodes 922. Although not shown in FIG. 9, IMD 12 may include switching circuitry configurable by processing circuitry 902 to control which of electrodes 922 is connected to therapy delivery circuitry 906 and sensing circuitry 904.

Sensation management circuitry 918 is configured to generate and deliver electrical therapy to tissue proximate the ribs of a patient (e.g., to reduce a sensation in one or more intercostal nerves). Sensation management circuitry 918 may include one or more pulse generators, capacitors, and/or other components capable of generating and/or storing energy to deliver as peripheral nerve stimulation, high frequency stimulation, irreversible electroporation, or another form of therapy.

Sensation management circuitry 918 may be configured to generate and deliver electrical therapy in response to one or more triggers or based on feedback from therapy delivery circuitry 906 or sensing circuitry 904. For example, sensation management circuitry 918 may be configured to start or alter therapy (e.g., automatically, without additional user input) to one or more intercostal nerves in response to therapy delivery circuitry 906 generating and delivering therapy to the heart. Because therapy delivered to a heart of a patient via therapy delivery circuitry 906 may cause a sensation in one or more intercostal nerves, automatic therapy delivery to one or more intercostal nerves may otherwise limit or prevent sensation that a patient would otherwise experience. For example, when therapy delivery circuitry 906 delivers therapy to the heart of the patient, sensation management circuitry 918 may increase the amplitude, frequency, or alter another parameter associated with electrical therapy delivered to one or more intercostal nerves.

While the example of FIG. 9 shows therapy delivery circuitry 906 and sensation management circuitry 918 as separate blocks connected to separate subsets of electrodes, therapy delivery circuitry 906 and sensation management circuitry 918 may be wholly or partially integrated. In some examples, therapy delivery circuitry 906 and sensation management circuitry 918 may perform their respective functions via common electrodes (such as electrodes 922 or electrodes 926). In this manor, some or all of electrodes 922 and/or electrodes 926 may be operatively connected to both therapy delivery circuitry 906 and sensation management circuitry 918.

Communication circuitry 910 may include any suitable hardware, firmware, software or any combination thereof for communicating with another device, such as a clinician programmer, a patient monitoring device, or the like. For example, communication circuitry 910 may include appropriate modulation, demodulation, frequency conversion, filtering, and amplifier components for transmission and reception of data with the aid of an antenna.

The various components of IMD 12 may include any one or more processors, controllers, digital signal processors (DSPs), application specific integrated circuits (ASICs), field-programmable gate arrays (FPGAs), or equivalent discrete or integrated circuitry, including analog circuitry, digital circuitry, or logic circuitry. Processing circuitry 902 may include fixed function circuitry and/or programmable processing circuitry. The functions attributed to processing circuitry 902 herein may be embodied as software, firmware, hardware or any combination thereof.

Memory 912 may include computer-readable instructions that, when executed by processing circuitry 902 or other components of IMD 12, cause one or more components of IMD 12 to perform various functions attributed to those components in this disclosure. Memory 912 may include any volatile, non-volatile, magnetic, optical, or electrical media, such as a random-access memory (RAM), read-only memory (ROM), non-volatile RAM (NVRAM), static non-volatile RAM (SRAM), electrically-erasable programmable ROM (EEPROM), flash memory, or any other non-transitory computer-readable storage media.

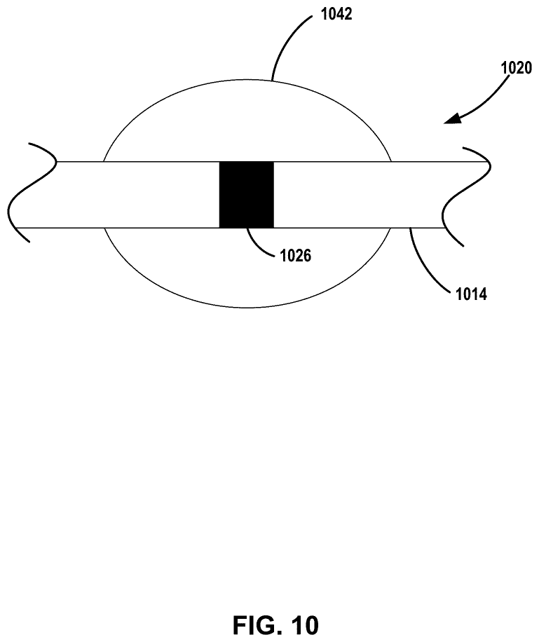

FIG. 10 is a conceptual diagram of a portion of a lead 1014 carrying a shield 1042 in accordance with techniques of this disclosure. In some examples, a distal portion 1020 of lead body 1014 may include one or more shields 1042. Lead 1014 may include one or more electrodes 1026, which may be an example of electrodes 26 as shown and described in connection with FIGS. 1-2. Shield 1042 may be configured to impede delivery of an electrical therapy via an electrode in a particular direction (e.g., in an anterior, posterior, superior, or inferior direction). For example, shield 1042 may be positioned relative to electrode 1026 to impede delivery of an electrical therapy via electrode 1026 in a direction from electrode 1026 toward an intercostal vascular bundle. Shield 1042 may be positioned between electrodes 1026 and one or more intercostal veins, intervascular arteries, or intercostal vascular bundles. As another example, shield 1042 may be positioned relative to electrodes 1026 to impede delivery of an electrical therapy via electrode 1026 in a direction from electrode 1026 toward the heart. Shield 1042 may be positioned between electrodes 1026 and the heart.

Shield 1042 may be electrically insulative. In some examples, shield 1042 may comprise a polymer, such as polyurethane. In some examples, shield 1042 may be configured to be folded or wrapped around electrodes 1026 for delivery via a lumen of an implant tool, and configured to elastically unfold or unwrap to a relaxed condition, e.g., such as the condition shown in FIG. 10, when released from the lumen. In some examples, shield 1042 may comprise elastic or super-elastic polymer or metallic structures, e.g., Nitinol structures, to encourage the deployment of shield 1042, support articulation of shield 1042, and/or support shield 1042 in the deployed, relaxed configuration. The deployed and/or articulated configuration may be substantially planar, as illustrated in FIG. 10, or may be non-planar. For example, portions of shield 1042 may include a cup shape or a bowl shape.

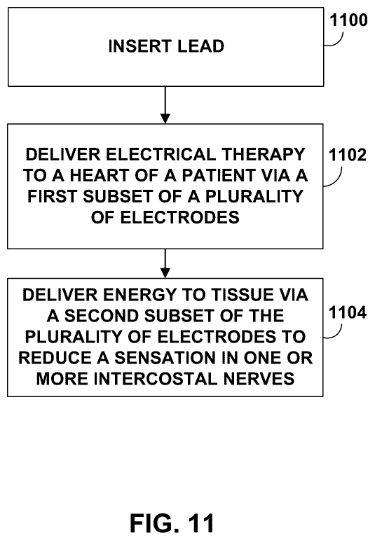

FIG. 11 is a flow diagram illustrating an example technique for using system 10 according to the techniques of this disclosure. The technique of FIG. 11 may be used with any of the devices or systems in connection with FIGS. 1-10, but is described with respect to system 10 in FIG. 1-2. Lead 14 (which may be an elongated structure) may be inserted into patient 13 and navigated or extended to an implantation site (1100). In some examples, lead 14 extends from an access point of patient 13 through intercostal space 31. In some examples, lead extends under ribs 15 (e.g., substernally). In some examples, lead 14 is navigated through patient 13 via an access tool or a delivery tool.

Continuing with the example of FIG. 11, the technique may further include delivering, by IMD 12 via electrodes 22 (which may be a first subset of electrodes), electrical therapy to heart 19 of patient 13 (1102). As described above, therapy delivered to heart 19 may include deliver cardiac pacing, such as bradycardia pacing, asystole pacing, anti-tachycardia therapy (ATP) pacing, post-shock pacing, or cardiac resynchronization therapy (CRT) pacing, or anti-tachyarrhythmia, e.g., cardioversion/defibrillation, shock to heart 19.

Continuing with the example of FIG. 11, the technique may further include delivering, by IMD 12 via electrodes 26 (which may be a second subset of electrodes), energy to tissue proximate ribs 15 of patient 13 to reduce a sensation in one or more intercostal nerves (1104). In some examples, delivery of energy to tissue proximate ribs 15 of patient 13 to reduce a sensation in one or more intercostal nerves occurs before delivery of electrical therapy to heart 19. In some examples, delivery of energy to tissue proximate ribs 15 of patient 13 to reduce a sensation in one or more intercostal nerves may additionally or alternatively occur concurrently with delivery of electrical therapy to heart 19. In some examples, delivery of energy to tissue proximate ribs 15 of patient 13 to reduce a sensation in one or more intercostal nerves may additionally or alternatively occur after delivery of electrical therapy to heart 19. In some examples, delivering energy to tissue proximate ribs 15 includes peripheral nerve stimulation or electroporation energy anterior to the heart to reduce pain or other sensation associated with navigation of lead 14 through intercostal space 31. Electrodes 26 may be disposed on lead 14 and may remain implanted, however electrodes may additionally or alternatively be disposed on an implant or delivery device and removed from patient 13 after an implant procedure. The technique may further include delivering a therapy agent (as described above in connection with FIG. 6 and FIGS. 7A-7F) to tissue proximate ribcage 15 of patient 13 via distal portion 20 of lead 14.

Example 1. A medical device comprising: an elongated structure configured to extend from an access point of a patient through an intercostal space within the patient; and a plurality of electrodes comprising a first subset of electrodes and a second subset of electrodes, wherein at least the first subset of electrodes is carried on a distal portion of the elongated structure, wherein the first subset of electrodes of the plurality of electrodes is configured to deliver electrical therapy to a heart of the patient or sense cardiac activity, and wherein the second subset of electrodes of the plurality of electrodes is configured to deliver energy to tissue proximate a ribcage of the patient to reduce a sensation in one or more intercostal nerves.

Example 2. The medical device of example 1, wherein to deliver energy to tissue proximate the ribcage of the patient, the second subset of electrodes is configured to deliver at least one of peripheral nerve stimulation or electroporation energy anterior to the heart to reduce pain associated with extension of the elongated structure through the intercostal space.

Example 3. The medical device of any of examples 1 to 2, wherein the second subset of electrodes comprises one or more segmented electrodes.

Example 4. The medical device of any of examples 1 to 3, wherein the second subset of electrodes comprises one or more coil electrodes.

Example 5. The medical device of any of examples 1 to 4, wherein at least a portion of the second subset of electrodes is configured to be oriented toward the intercostal space.

Example 6. The medical device of any of examples 1 to 5, wherein the first subset of electrodes and the second subset of electrodes share at least one common electrode.

Example 7. The medical device of any of examples 1 to 6, wherein the second subset of electrodes is configured to remain implanted after the elongated structure is navigated through the patient.

Example 8. The medical device of any of examples 1 to 7, wherein the second subset of electrodes is carried by the elongated structure.

Example 9. The medical device of any of examples 1 to 8, wherein the second subset of electrodes is carried by the elongated structure such that the second subset of electrodes is positioned in the intercostal space when implanted within the patient.

Example 10. The medical device of any of examples 1 to 6, wherein the second subset of electrodes is carried by a medical device housing, the medical device housing configured to couple to the elongated structure.

Example 11. The medical device of any of examples 1 to 6, wherein the second subset of electrodes is carried by a delivery device, wherein the delivery device is configured to deliver the elongated structure to an implant location within the patient.

Example 12. The medical device of any of examples 1 to 6, wherein the elongated structure is a first elongated structure, wherein the medical device further comprises: a second elongated structure, wherein the second subset of electrodes is carried on the second elongated structure.

Example 13. The medical device of example 12, wherein the second elongated structure branches from the first elongated structure.

Example 14. The medical device of any of examples 1 to 13, wherein the tissue comprises at least one of muscle tissue or nerve tissue.

Example 15. The medical device of any of examples 1 to 14, wherein to deliver electrical therapy to the heart of the patient, the first subset of electrodes is configured to provide one or more of pacing or cardioversion/defibrillation shock to the heart of the patient.

Example 16. The medical device of any of examples 1 to 15, wherein the distal portion of the elongated structure is configured to deliver a therapy agent to tissue proximate the ribcage of the patient, and wherein the therapy agent comprises at least one of an anti-inflammatory agent, an analgesic agent, a neurotoxin, or an antimicrobial agent.

Example 17. The medical device of any of examples 1 to 16, wherein the elongated structure is a catheter, an introducer, or an implantable medical lead.

Example 18. The medical device of any of examples 1 to 17, further comprising a shield configured to impede delivery of energy from the second subset of electrodes toward the heart.

Example 19. The medical device of any of examples 1 to 18, further comprising a shield configured to impede delivery of energy from the second subset of electrodes toward an intercostal vascular bundle.

Example 20. The medical device of any of examples 1 to 19, wherein the first subset of electrodes comprises one or more coil electrodes configured to deliver defibrillation therapy, and wherein the first subset comprises one or more electrodes configured to deliver pacing to the heat or sense cardiac activity.

Example 21. A method comprising: delivering, by a medical device and via a first subset of a plurality of electrodes, electrical therapy to a heart of a patient, wherein the first subset of electrodes of the plurality of electrodes is carried by a distal portion of an elongated structure extending from an access point of the patient through an intercostal space within the patient; and delivering, by the medical device and via a second subset of the plurality of electrodes of the medical device, energy to tissue proximate the ribcage of the patient to reduce a sensation in one or more intercostal nerves.

Example 22. The method of example 21, wherein delivering energy to tissue proximate the ribcage of the patient comprises delivering at least one of peripheral nerve stimulation or electroporation energy anterior to the heart to reduce pain associated with extension of the elongated structure through the intercostal space.

Example 23. The method of any of examples 21 to 22, wherein the second subset of electrodes comprises one or more segmented electrodes.

Example 24. The method of any of examples 21 to 23, wherein the second subset of electrodes comprises one or more coil electrodes.

Example 25. The method of any of examples 21 to 24, wherein at least a portion of the second subset of electrodes is configured to be oriented toward the intercostal space.

Example 26. The method of any of examples 21 to 25, wherein the first subset of electrodes and the second subset of electrodes share at least one common electrode.

Example 27. The method of any of examples 21 to 26, wherein the second subset of electrodes is configured to remain implanted after the elongated structure is navigated through the patient.

Example 28. The method of any of examples 21 to 27, wherein the second subset of electrodes is carried by the elongated structure.

Example 29. The method of any of examples 21 to 28, wherein the second subset of electrodes is carried by a delivery device, wherein the delivery device is configured to deliver the elongated structure to an implant location within the patient.

Example 30. The method of any of examples 21 to 29, wherein the elongated structure is a first elongated structure, wherein the medical device further comprises: a second elongated structure, wherein the second subset of electrodes is carried on the second elongated structure.

Example 31. The method of example 30, wherein the second elongated structure branches from the first elongated structure.

Example 32. The method of any of examples 21 to 31, wherein the tissue comprises at least one of muscle tissue or nerve tissue.

Example 33. The method of any of examples 21 to 32, wherein delivering electrical therapy to the heart of the patient comprises providing one or more of pacing or cardioversion/defibrillation shock to the heart of the patient.

Example 34. The method of any of examples 21 to 33 further comprising delivering a therapy agent to tissue proximate a ribcage of the patient via the distal portion of the elongated structure, wherein the therapy agent comprises at least one of an anti-inflammatory agent, an analgesic agent, a neurotoxin, or an antimicrobial agent.

Example 35. The method of any of examples 21 to 34, wherein the elongated structure is a catheter, an introducer, or an implantable medical lead.

Example 36. A medical device comprising: an elongated structure configured to extend from an access point of a patient through an intercostal space within the patient; and a plurality of electrodes carried on a distal portion of the elongated structure, wherein at least a subset of electrodes of the plurality of electrodes is configured to at least deliver electrical therapy to a heart of the patient or sense cardiac activity, wherein the distal portion of the elongated structure is configured to deliver a therapy agent to tissue proximate a ribcage of the patient, and wherein the therapy agent comprises at least one of an anti-inflammatory agent, an analgesic agent, a neurotoxin, or an antimicrobial agent.

Example 37. The medical device of example 36, wherein the therapy agent is carried by the distal portion of the elongated structure.

Example 38. The medical device of any of examples 36 to 37, wherein the therapy agent is carried by the elongated structure such that the therapy agent is positioned in the intercostal space when implanted within the patient.

Example 39. The medical device of any of examples 36 to 38, wherein the therapy agent is disposed on the distal portion of the elongated structure.

Example 40. The medical device of any of examples 36 to 39, wherein the elongated structure comprises a coating, and wherein the coating comprises the therapy agent.

Example 41. The medical device of any of examples 36 to 40, wherein the elongated structure comprises a sleeve disposed over the distal portion of the elongated structure, and wherein the sleeve comprises the therapy agent.

Example 42. The medical device of any of examples 36 to 41, wherein the elongated structure comprises a mesh disposed proximate the distal portion of the elongated structure, and wherein the mesh comprises the therapy agent.

Example 43. The medical device of any of examples 36 to 42, wherein the elongated structure comprises a resorbable material disposed proximate the distal portion of the elongated structure, and wherein the resorbable material comprises the therapy agent.

Example 44. The medical device of any of examples 36 to 43, wherein the therapy agent is incorporated into a body of the elongated structure.

Example 45. The medical device of any of examples 36 to 44, wherein the therapy agent is eluted from the elongated structure via a reservoir.

Example 46. The medical device of any of examples 36 to 45, wherein the subset of electrodes of the plurality of electrodes configured to at least deliver electrical therapy to the heart of the patient or sense cardiac activity is a first subset of electrodes, and wherein the medical device further comprises: a second subset of electrodes of the plurality of electrodes configured to deliver energy to tissue proximate the ribcage of the patient.

Example 47. The medical device of any of examples 36 to 46, wherein to deliver energy to tissue proximate the ribcage of the patient, the second subset of electrodes is configured to deliver at least one of peripheral nerve stimulation or electroporation energy to reduce a sensation in one or more intercostal nerves.

Example 48. The medical device of any of examples 36 to 47, wherein the elongated structure is a catheter, an introducer, or an implantable medical lead.

1. A medical device comprising:
an elongated structure configured to extend from an access point of a patient through an intercostal space within the patient; and
a plurality of electrodes comprising a first subset of electrodes and a second subset of electrodes,
wherein at least the first subset of electrodes is carried on a distal portion of the elongated structure, wherein the first subset of electrodes of the plurality of electrodes is configured to deliver electrical therapy to a heart of the patient or sense cardiac activity, and wherein the second subset of electrodes of the plurality of electrodes is configured to deliver energy to tissue proximate a ribcage of the patient to reduce a sensation in one or more intercostal nerves.

2. The medical device of claim 1, wherein to deliver energy to tissue proximate the ribcage of the patient, the second subset of electrodes is configured to deliver at least one of peripheral nerve stimulation or electroporation energy anterior to the heart to reduce pain associated with extension of the elongated structure through the intercostal space.

3. The medical device of claim 1, wherein at least a portion of the second subset of electrodes is configured to be oriented toward the intercostal space.

4. The medical device of claim 1, wherein the first subset of electrodes and the second subset of electrodes share at least one common electrode.

5. The medical device of claim 1, wherein the second subset of electrodes is configured to remain implanted after the elongated structure is navigated through the patient.

6. The medical device of claim 1, wherein the second subset of electrodes is carried by the elongated structure.

7. The medical device of claim 1, wherein the second subset of electrodes is carried by the elongated structure such that the second subset of electrodes is positioned in the intercostal space when implanted within the patient.

8. The medical device of claim 1, wherein the second subset of electrodes is carried by a medical device housing, the medical device housing configured to couple to the elongated structure.

9. The medical device of claim 1, wherein the second subset of electrodes is carried by a delivery device, wherein the delivery device is configured to deliver the elongated structure to an implant location within the patient.

10. The medical device of claim 1, wherein the elongated structure is a first elongated structure, wherein the medical device further comprises:
a second elongated structure, wherein the second subset of electrodes is carried on the second elongated structure.

11. The medical device of claim 1, wherein the tissue comprises at least one of muscle tissue or nerve tissue.

12. The medical device of claim 1, wherein to deliver electrical therapy to the heart of the patient, the first subset of electrodes is configured to provide one or more of pacing or cardioversion/defibrillation shock to the heart of the patient.

13. The medical device of claim 1, wherein the distal portion of the elongated structure is configured to deliver a therapy agent to tissue proximate the ribcage of the patient, and wherein the therapy agent comprises at least one of an anti-inflammatory agent, an analgesic agent, a neurotoxin, or an antimicrobial agent.

14. The medical device of claim 1, further comprising a shield configured to impede delivery of energy from the second subset of electrodes toward the heart.

15. The medical device of claim 1, wherein the first subset of electrodes comprises one or more coil electrodes configured to deliver defibrillation therapy, and wherein the first subset comprises one or more electrodes configured to deliver pacing to the heat or sense cardiac activity.

16. The medical device of claim 1, wherein the distal portion of the elongated structure is configured to deliver a therapy agent to tissue proximate the ribcage of the patient, and wherein the therapy agent comprises at least one of an anti-inflammatory agent, an analgesic agent, a neurotoxin, or an antimicrobial agent.

17. The medical device of claim 1, wherein the elongated structure is a catheter, an introducer, or an implantable medical lead.

18. The medical device of claim 1, further comprising a shield configured to impede delivery of energy from the second subset of electrodes toward the heart or an intercostal vascular bundle.

19. The medical device of claim 1, wherein the first subset of electrodes comprises one or more coil electrodes configured to deliver defibrillation therapy, and wherein the first subset comprises one or more electrodes configured to deliver pacing to the heat or sense cardiac activity.

20. A medical device comprising:
a cardiac lead configured to extend from an access point of a patient through an intercostal space within the patient; and
a plurality of electrodes carried on a distal portion of the lead, wherein at least a subset of electrodes of the plurality of electrodes is configured to at least deliver electrical therapy to a heart of the patient or sense cardiac activity, wherein the distal portion of the lead is configured to deliver a therapy agent to tissue proximate a ribcage of the patient, and wherein the therapy agent comprises at least one of an anti-inflammatory agent, an analgesic agent, a neurotoxin, or an antimicrobial agent.
